(19) United States
(12) Patent Application Publication
(10) Pub. No.: US 2011/0176106 A1
(43) Pub. Date: Jul. 21, 2011

(54) PORTABLE EYE MONITORING DEVICE AND METHODS FOR USING THE SAME

(76) Inventor: Christopher Paul Lewkowski, Western Australia (AU)

(21) Appl. No.: 12/299,222

(22) PCT Filed: Apr. 30, 2007
(86) PCT No.: PCT/AU07/00557

§ 371 Date: Feb. 20, 2009

(30) Foreign Application Priority Data
May 1, 2006 (AU) 2006902239

(51) Int. Cl. A61B 3/14

(52) U.S. Cl. 351/206; 351/246

(57) ABSTRACT

A portable device in the form of a mask (10) that can be worn over a user's eyes (22) is provided with a non-visible light source to illuminate the user's eyes (22), and a lens arrangement (30) and image capturing means to capture digital images eyes (22) that can then be processed and used to diagnose a variety of dizziness and balance-related disorders and disease. The mask (10) includes a light-omitting seal or cover (16) to prevent external light reaching the eyes (22). The images can then be sent via data transmitter to an external device for processing and/or further analysis. The mask (10) has particular suitability for use remotely by patients themselves during a dizziness episode.

### FIELD OF THE INVENTION

The present invention relates to the investigation of changes in the eye and relates particularly, though not exclusively, to a portable device for the investigation of nystagmus which may be associated with vestibular and other neurological disorders, and a method of conducting the investigation using the device.

Throughout the specification, unless the context requires otherwise, the word “comprise” or variations such as “comprises” or “comprising”, will be understood to imply the inclusion of a stated integer or group of integers but not the exclusion of any other integer or group of integers.

### BACKGROUND ART

The following discussion of the background art is intended to facilitate an understanding of the invention. However, it should be appreciated that the discussion is not an acknowledgement or admission that any of the material referred to was published, known or part of the common general knowledge of the person skilled in the art as at the priority date of the application.

The presence of a number of medical and health problems can be detected by assessing the eye, including unusual changes in the position, movement and dilation of the eye. For example, it has been found that one of the leading causes of dizziness and balance problems is associated with the vestibular system in the inner ear. The presence of nystagmus (very specific, rapid, involuntary eye movements) during a dizziness attack can suggest to a clinician that there is a vertiginous component to it and the direction of the nystagmus may provide some evidence to a specialist in the field, of more specific information, such as which ear (or which part of the ear) has the active disease.

Dizziness and balance problems constitute a major public health problem. A significant proportion of adults have had an episode of dizziness that occurs with enough intensity or frequency to promote a visit to the doctor. Accurate diagnosis of balance symptoms is important not only to exclude potentially serious central causes but to aid successful treatment. Unfortunately diagnosis is sometimes not possible, or delayed. Studies have shown that general practitioners (GPs) rarely failed to refer urgent cases but often failed to refer patients with persistent vestibular conditions.

Dizziness is one of the most difficult complaints to assess, as it is a subjective symptom of potentially numerous causes. Dizziness is often an episodic symptom, with the frequency of episodes highly variable. Often the patients present to the healthcare professional with no symptoms as they have no symptoms during their appointment. As a patient will very rarely have an episode in the clinic, clinicians are frequently forced to rely solely on the patient's (often unintentionally misleading) subjective interpretation of the symptom. There are unfortunately very few objective measures of the symptom of dizziness.

The presence of nystagmus (very specific eye movements) during an attack can tell a clinician that there is a vertiginous component to a dizziness attack and may provide some evidence to a specialist in the field of more specific information, such as which ear has the active disease. Part of the difficulty is that prior art nystagmography apparatus for detecting and recording nystagmus are large, non-portable, expensive machines that are only available for use by specialists. Due to the size, complexity and expense of these machines they are limited to laboratory use for specific tests. For this reason, these machines are rarely used to monitor patients at the time of a dizzy attack.

Furthermore, current devices require the image capturing component (goggles) to be linked to a data processing unit by wires, i.e. computer station or laptop, and are often powered from an external source. This prevents the use of these devices by the patient in a location remote from the clinic or without the assistance of another person or medical practitioner. It also limits the movement of the patient during examination, which makes a number of physical movements of the patient by the practitioner difficult or impossible.

The present invention was developed with a view to providing a portable device for the investigation of nystagmus and a method of conducting the investigation using the device. The device can be used away from the clinic and does not need a specialist to operate it. However it will be understood that the device may also be used more generally in oculography and for the investigation of other changes in the eye.

### DISCLOSURE OF THE INVENTION

In accordance with a first aspect of the present invention, there is provided a portable device adapted to cover the eyes of a subject in a manner that omits visible light, the device comprising:
(i) light omission means arranged to substantially reduce visible light stimulation of the subject's eyes;
(ii) a non-visible light generating means to illuminate the subject's eyes;
(iii) a lens member for creating images of the subjects eye; and
(iv) an image capturing means for capturing the images in digital form.

Preferably the non-visible light is infrared light.

Preferably, the light omission means comprises a light proof seal.

Preferably, the lens member has a depth of field sufficient to accommodate different distances between various subject's eyes and the lens member.

Preferably, the lens member has a fixed depth of field. Alternatively, the lens member may be adjustable.

Preferably, the device comprises a plurality of lens members. Preferably, the device comprises two lens members, one for each eye.

Preferably, the image capturing means is a digital image sensor. Preferably, the device comprises two digital image sensors, one for each eye. Preferably, the digital image sensor is capable of capturing at least 50-60 images per second.

Further, the digital image sensor may be capable of capturing at least 100-500 images per second.

Preferably, the digital image sensor is a charge coupled device. Alternatively, the digital image sensor is a complementary metal oxide semiconductor device.

Preferably, the lens member and the image capturing means are provided integrally. When provided integrally they may be provided in the form of a camera.

Preferably, when the device includes two integral lens members and image capturing means, such as two cameras, one for each eye, they may be adapted to be synchronised to record images of both eyes at the same time.

Preferably, the device has an integral power source.

Preferably, the integral power source includes an electrical cell.

Alternatively, the device is arranged to be coupled to a separate power source.

Preferably, the device is of a size that enables it to be conveniently handheld.

Preferably, the device further includes a data processing means for processing the captured images.

Preferably, the data processing means includes a processing unit.

Preferably, the data processing means includes two processing units, one for each eye.

Preferably, the two processing units are operable in a Master/Slave configuration for processing the images from the left and right eye respectively.

Preferably, the data processing means includes a tagging function to allocate a marker to the stored images.

Preferably, the data processing means may further comprise a communication means to communicate the status of the data processing means and/or the images contained therein.

Preferably, the communication means includes an audio means for providing an audible indication to a user.

Preferably, the communication means includes a visual display.

Preferably, the communication means includes a user control interface.

Preferably, the user control interface and the visual display are provided integrally such as in a liquid crystal display (LCD) touch screen or the like.

Preferably, the data processing means includes a reporting function operable to provide reports based on interrogation of data processed by the data processing means.

Preferably, the device further includes a data transmitting means for transmitting the captured images to a separate device.

Preferably, the data transmitting means is a wireless data transmitter. Alternatively, the data transmitting means is a wired data transmitter.

Preferably, the data transmitting means is operable to transmit via the Internet.

Preferably, the data transmitting means is operable to transmit in real-time.

Preferably, the device further includes a data processing means for processing and storing the captured images, and data transmitting means operable to transmit the captured and stored images some time after they are captured.

Preferably, the portable device further includes a data storage means for storing captured images thereon. Preferably, the data storage means is a removable storage device. Alternatively, the data storage means is fixed within the portable device.

In accordance with a second aspect of the present invention, there is provided a method of assessing eye movement using a portable device in accordance with an aspect of the present invention as herein described, the method comprising the steps of:
(i) applying the device to a subject when changes in eye movement are present; and
(ii) capturing images of the subject's eyes in digital form.

Preferably, the method includes the further steps of: applying the device to the subject, at a first location, when changes in the eye may be present; and transmitting the images to a medical professional, at a second location, to assess the presence of a disorder.

Preferably, the subject applies the device themselves during an episode in which changes in the eye may be present.

Preferably, the transmitted images are unprocessed and the method includes the step of processing the transmitted images remotely from the subject. Alternatively, the method includes the step of processing the images by the device prior to being sent to the medical professional.

Preferably, the method includes the step of producing a report and transmitting the report to the medical professional.

In accordance with a third aspect of the present invention, there is provided a method of monitoring the effectiveness of a treatment for dizziness or balance related disorder using a portable device in accordance with an aspect of the present invention as herein described, the method comprising the steps of:
(i) applying the device to a subject undergoing treatment for the disorder when changes in eye movement are present;
(ii) capturing images of the subject's eyes in digital form; and
(iii) analysing the images to monitor the effectiveness of the treatment.

Preferably, the step of analysing the images includes comparing the images to images of the same subject's eyes taken prior to treatment or earlier in the treatment regime when changes in the eye movement were present.

In accordance with a fourth aspect of the present invention, there is provided a method of monitoring the effects of a treatment capable of causing dizziness or balance related disorder using a portable device in accordance with an aspect of the present invention as herein described, the method comprising the steps of:
(i) applying the device to a subject undergoing treatment when changes in eye movement are present;
(ii) capturing images of the subject's eyes in digital form; and
(iii) analysing the images to monitor the effectiveness of the treatment.

Preferably, the step of analysing the images comprises comparing the images to images of the same subject's eyes taken prior to treatment or earlier in the treatment regime when changes in the eye movement were present.

In accordance with a fifth aspect of the present invention, there is provided a system for the bedside monitoring of patients, the system involving the use of a portable device in accordance with an aspect of the present invention as herein described.

In accordance with a sixth aspect of the present invention, there is provided a system for the monitoring of patients in an emergency room, the system involving the use of a portable device in accordance with an aspect of the present invention as herein described.

In accordance with a seventh aspect of the present invention, there is provided a database containing an eye movement data correlated with a range of disorders and/or conditions.

In accordance with an eighth aspect of the present invention, there is provided a method of diagnosing a disease or disorder correlated with eye movement in a subject using a portable device in accordance with an aspect of the present invention as herein described, the method comprising the steps of:
(i) capturing images of the subject's eyes in digital form using the device; and
(ii) comparing said images with a database of eye movement data correlated with a range of disorders and/or conditions to diagnose the disease or disorder.

In accordance with a ninth aspect of the present invention, there is provided a portable device adapted to cover the eyes of a subject in a manner that omits visible light, the device comprising:
(i) a non-visible light generating means to illuminate the subject's eyes;
(ii) a lens member for creating clear images of the subjects eye;
(iii) an image capturing means for capturing the images in digital form; and
(iv) a data processing means for processing the captured images.

In accordance with a tenth aspect of the present invention, there is provided a portable device adapted to cover the eyes of a subject in a manner that omits visible light, the device comprising:
(i) a non-visible light generating means to illuminate the subject's eyes;
(ii) a lens member for creating clear images of the subjects eye;
(iii) an image capturing means for capturing the images in digital form; and
(iv) a data transmitting means for transmitting the captured images to a separate device.

In accordance with an eleventh aspect of the present invention, there is provided a portable device adapted to cover the eyes of a subject in a manner, that omits visible light, the device comprising:
(i) a non-visible light generating means to illuminate the subject's eyes;
(ii) a lens member for creating clear images of the subjects eye;
(iii) an image capturing means for capturing the images in digital form;
(iv) a data processing means for processing the captured images; and
(v) a data transmitting means for transmitting the captured images to a separate device.

In accordance with a twelfth aspect of the present invention, there is provided a method of assessing eye movement using a portable device described herein. The method may comprise the steps of:
(i) applying the device to a subject when changes in eye movement are present; and
(ii) capturing images of the subject's eyes in digital form.

In accordance with a thirteenth aspect of the present invention, there is provided a portable device adapted to cover the eyes of a subject in a manner that omits visible light, the device consisting essentially of:
(i) light omission means arranged to substantially reduce visible light stimulation of the subject's eyes;
(ii) a non-visible light generating means to illuminate the subject's eyes;
(iii) a lens member for creating images of the subjects eye; and
(iv) an image capturing means for capturing the images in digital form.

In accordance with a fourteenth aspect of the present invention, there is provided a method of assessing eye movement using a portable device in accordance an aspect of the present invention as herein described, the method consisting essentially of:
(i) applying the device to a subject when changes in eye movement are present; and
(ii) capturing images of the subject's eyes in digital form.

In accordance with a fifteenth aspect of the present invention, there is provided a method of monitoring the effectiveness of a treatment for dizziness or balance related disorder using a portable device in accordance with an aspect of the present invention as herein described, the method consisting essentially of:
(i) applying the device to a subject undergoing treatment for the disorder when changes in eye movement are present;
(ii) capturing images of the subject's eyes in digital form; and
(iii) analysing the images to monitor the effectiveness of the treatment.

In accordance with a sixteenth aspect of the present invention, there is provided a method of monitoring the effects of a treatment capable of causing dizziness or balance related disorder using a portable device in accordance with an aspect of the present invention as herein described, the method consisting essentially of:
(i) applying the device to a subject undergoing treatment when changes in eye movement are present;
(ii) capturing images of the subject's eyes in digital form; and
(iii) analysing the images to monitor the effectiveness of the treatment.

In accordance with a seventeenth aspect of the present invention, there is provided a system for the bedside monitoring of patients, the system consisting essentially of the use of a portable device in accordance with an aspect of the present invention as herein described.

In accordance with an eighteenth aspect of the present invention, there is provided a system for the monitoring of patients in an emergency room, the system consisting essentially of the use of a portable device in accordance with an aspect of the present invention as herein described.

In accordance with a nineteenth aspect of the present invention, there is provided a database consisting essentially of an eye movement data correlated with a range of disorders and/or conditions.

In accordance with a twentieth aspect of the present invention, there is provided a method of diagnosing a disease or disorder correlated with eye movement in a subject using a portable device in accordance with an aspect of the present invention as herein described, the method consisting essentially of:
(i) capturing images of the subject's eyes in digital form using the device; and
(ii) comparing said images with a database of eye movement data correlated with a range of disorders and/or conditions to diagnose the disease or disorder.

In accordance with a twenty-first aspect of the present invention, there is provided a portable device adapted to cover the eyes of a subject in a manner that omits visible light, the device consisting essentially of:
(i) a non-visible light generating means to illuminate the subject's eyes;
(ii) a lens member for creating clear images of the subjects eye;
(iii) an image capturing means for capturing the images in digital form; and
(iv) a data processing means for processing the captured images.

In accordance with a twenty-second aspect of the present invention, there is provided a portable device adapted to cover the eyes of a subject in a manner that omits visible light, the device consisting essentially of:
(i) a non-visible light generating means to illuminate the subject's eyes;
(ii) a lens member for creating clear images of the subjects eye;
(iii) an image capturing means for capturing the images in digital form; and
(iv) a data transmitting means for transmitting the captured images to a separate device.

In accordance with a twenty-third aspect of the present invention, there is provided a portable device adapted to cover the eyes of a subject in a manner that omits visible light, the device consisting essentially of:
(i) a non-visible light generating means to illuminate the subject's eyes;
(ii) a lens member for creating clear images of the subjects eye;
(iii) an image capturing means for capturing the images in digital form;
(iv) a data processing means for processing the captured images; and
(v) a data transmitting means for transmitting the captured images to a separate device.

In accordance with a twenty-fourth aspect of the present invention, there is provided a method of assessing eye movement using a portable device described herein. The method may consist essentially of:
(i) applying the device to a subject when changes in eye movement are present; and
(ii) capturing images of the subject's eyes in digital form.

The portable device has the advantage that it has a functionality and data output comparable to much larger and more expensive devices that are currently used in this area. Importantly, the device records images digitally and is portable so it can be used to record eye movements during actual dizziness attacks suffered by subjects. Furthermore, the device of the present invention can be conveniently fitted and used by subjects outside of a hospital or clinical environment as the device is simple to use and does not have to be physically connected to other less portable devices such as computers or other apparatus. The device may also enjoy one or more of the following advantages: lightweight, durable and inexpensive to manufacture.

### BRIEF DESCRIPTION OF THE DRAWINGS

The nature of the invention will be better understood from the following detailed description of preferred embodiments of the device and method of using the same, given by way of example only, with reference to the accompanying drawings, in which:

FIG. 1 is a schematic diagram illustrating the basic principles of a device in accordance with an aspect of the present invention;

FIG. 2 illustrates a preferred embodiment of the device in accordance with an aspect of the present invention;

FIG. 3 is a functional block diagram of the internal electronic components in the device mask of FIG. 2; and

FIG. 4 is a flow chart illustrating a preferred method of using the device of FIG. 2.

### BEST MODE(S) FOR CARRYING OUT THE INVENTION

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

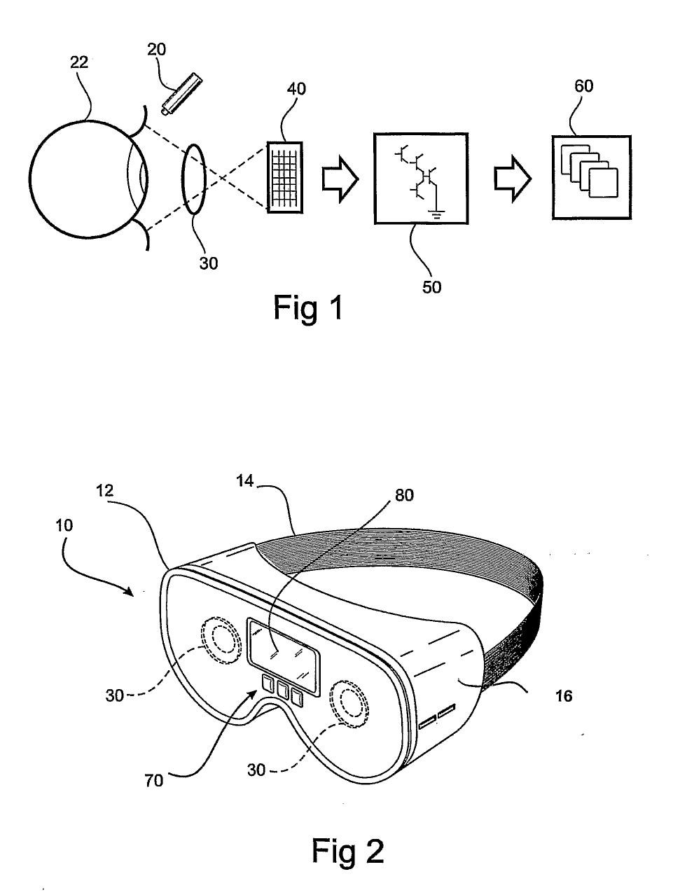

A preferred embodiment of a portable device in the form of a mask 10 according to the present invention, as shown in FIGS. 1 and 2, comprises a lightweight, robust, plastics or metal frame or body 12 on which the other components are supported. For the purposes of the present invention “portable” means that the device is adapted for use outside of a hospital or clinic environment and does not require a physical connection with another device, such as a data processor, computer or storage device, to enable it to operate. In one embodiment, the portable device is of a size that enables it to be conveniently handheld.

The body 12 preferably includes straps or arms 14 to allow the mask 10 to be temporarily attached to the user's head. Preferably the mask 10 is designed to provide a light omitting, tightly fitting cover 16 for the user's eyes 22. This is achieved by the provision of a light-proof seal which blocks out external light entering the mask 10, when worn by a user. In the embodiment described herein, the mask 10 is arranged to cover both eyes and therefore substantially blocks out external light from reaching the eyes. This is specifically advantageous for detecting nystagmus where it is useful to block out all visible light stimulation to the eyes, as the user could override the spontaneous nystagmus reflex by fixating on a point. Any suitable light-proof seal can be used, such as a moulded opaque rubber seal, for example.

The mask 10 may also include a seal indicating means that provides a suitable indication that the mask 10 is properly fitted. This indicating means may take the form of a light detecting electronic sensor that is operable to emit light or sound via a user control interface 70, described in further detail below, to indicate whether the mask 10 is properly fitted (i.e. no light sensed by the light detecting electronic sensor), or otherwise.

A light source 20 operating in a non-visible part of the electromagnetic spectrum is provided to illuminate the user's eyes 22. Any suitable frequency or frequencies in the non-visible part of the electromagnetic spectrum can be used: providing that it is suitable to be used with the human body. In a preferred embodiment the light source 20 is an infrared light source, as infrared is harmless to human tissue at low power levels and is widely used. The infrared light source 20 may be light emitting diodes (LEDs) mounted within the body 12, a set of one or many provided for each eye.

A pair of lenses 30—one for each of the user's eyes 22—is provided to focus clear images of the user's eyes 22 onto a pair of image capturing means in the form of digital image sensors 40. In the embodiment described herein, there is one digital image sensor 40 for each lens 30, although other arrangements could be used such as one large digital image sensor for both lenses 30. The factors to be considered in choosing a suitable lens are focal length, sensor size, distance to image plane, image size and aperture (light). One particular preferable requirement of the lens 30 is to have a depth of field that will allow the eye image to be always in focus. As different users will have varying length between the lens and eye, the lens will usefully have a depth of field over this range. Thus, the lenses 30 may have a fixed depth of field, or it may be adjustable, for example using auto-focusing technology or to allow the focus to be adjusted by the wearer and/or a clinician.

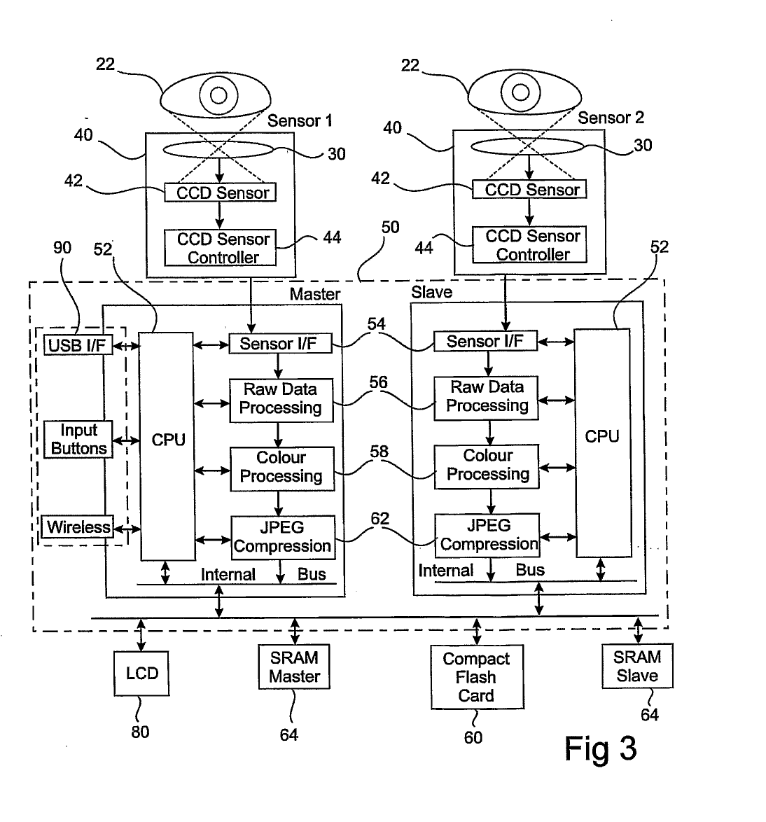

The digital image sensors 40 can be complementary metal-oxide semiconductor (CMOS) or charge coupled device (CCD) based image sensors. Such image sensors are well known to persons skilled in the art. Any other suitable imaging device can be used that captures optical/light signals and converts to electrical signals for subsequent processing. For a CCD sensor, each sensor 40 typically includes a CCD sensor 42 and a CCD sensor controller 44 as shown in FIG. 3. Preferably the lenses 30 are incorporated into or attached to the image sensors 40. The image sensors 40 are operated to capture images at a predefined frame rate. In this embodiment, the frame rate is at least 50 to 60 images per second and advantageously 100 to 500 images per second.

In one embodiment, the lenses 30 and the digital image sensors 40 are provided integral with the mask 10. When provided integrally, they can be provided in the form of a camera. If two cameras are used, then the cameras are adapted to synchronously record images from both eyes 30 at the same time as will be described further below.

The raw or unprocessed images captured by the image sensors 40 are transferred synchronously to a digital image processing means in the form of an image processing unit (IPU) 50. The IPU 50 may correct the images received for such photographic problems as over-exposure or under-exposure, bad pixels, colour variation or other image deficiencies and errors. This image processing is well known to persons skilled in the art, and, as such need not be described in any further detail herein, except as is relevant to the present invention.

In the embodiment described, the captured and processed image is then compressed by the IPU 50 using an industry standard compression algorithm. Currently this is the JPEG standard, however it is to be understood that any suitable compression algorithm may be employed.

In alternative embodiments of the invention, the image is not compressed.

Operation of the IPU 50 will now be described in more detail with reference to FIG. 3. The IPU 50 is responsible for coordinating the collection, manipulation, compression, sequencing and transfer of the images of both the user's eyes 22. It includes various electronic components that facilitate these functions. Preferably the IPU 50 employs dual central processing units (CPUs) 52 operated in a Master/Slave configuration for processing the left and right eye images respectively. Raw image data is received from the respective image sensors 40 via sensor I/F devices 54 and processed in raw data processing devices 56 and colour processing devices 58. The raw data processing devices 56 and colour processing devices 58 will adjust the images to compensate for light variations and colour matching. The processed images are then compressed using a compression device 62. The compression method may include JPEG, and any other suitable compression algorithms. Compression ratio is configurable based on clarity and speed requirements. The sequence of compressed images is then either stored within the mask's 10 internal memory, in this case static random access memory (SRAM) 64, or on a removable digital media device such as a compact flash (CF) card 60, or transferred to an external device via a data transmitting means in the form of a communications port 90.

If a removable storage device is used, the JPEG compressed images are written to the data storage means such as the CF card 60. In this embodiment, the CF card 60 is used as the storage medium but other types of storage media could be used. The main reason for using this memory type is that it is currently widely available and has a large memory capacity. It is also relatively large in physical dimensions and therefore easy to handle. This is important as elderly and technologically challenged wearers may be changing the memory device by themselves, for instance, to replace it with a fresh one when it is full. In a preferred embodiment, the data captured is rendered tamper-proof thus ensuring the integrity of the data with regards to accurate diagnosis and medical liability issues.

The SRAM 64 and the removable storage device such as the CF card 60 may also store unprocessed images. This would be done, for example, where the processing of the images is being done by a separate device, as will be discussed below.

In this way, the IPU 50 is operable to process the images independently, but because of the Master/Slave configuration the images can also be processed synchronously. This has the advantage in that it allows for increased accuracy in measuring eye movements. It also advantageously allows for the comparison of eye attributes such as pupil diameter, which may be indicative of particular diseases and disorders.

The IPU 50 may also employ a tagging function that enables data to be allocated a marker such as time or date and/or name of the user. This enables data from two eyes to be cross-referenced and also enables the data to be more easily analysed at a later date.

As discussed above, the images can be processed externally by a separate device. This may be a computer or other device operable to further process, view, store and interrogate the images using appropriate software. Thus, the mask 10 may not include an IPU 50, with the captured images being processed by the separate device.

If unprocessed images are to be processed externally (whether or not there is an IPU 50 provided in the mask 10), the mask 10 will include a data transmitting means such as a communications port 90 to transmit the images to the separate device for processing thereby. The communications port 90 can also be used to transmit processed images as will be discussed further below.

In a preferred embodiment, a communication means in the form of user control interface 70 is built into the mask 10 for inputting commands or instructions to the IPU 50 (see FIG. 1). This could include a keypad, a series of input switches or dials, or a touch screen. The user control interface 70 may also include a status indicator, which may consist of an LCD screen 80, LEDs, a speaker or other audio/sound device, which the IPU 50 can use to indicate its status and/or the quality of the recorded images to the user. The LCD screen 80 may permit the user, their spouse, or a clinician to easily ascertain whether or not the device is correctly recording the images of both eyes. In addition the user may view the stored images to confirm that the recording is correctly working, or a clinician may quickly review the images prior to downloading and transmission to a specialist laboratory for analysis. Furthermore, the user control interface 70 may be operable to enable a user to operate or otherwise control the IPU 50, and hence the mask 10.

As mentioned above, a data transmitting means can be provided on the mask 10. In this embodiment, the data transmitting means is in the form of wired and/or wireless communication port 90 built into the mask 10 for downloading images from the CF card 60, or SRAM 64, at a later time, or in real time as they are captured. Typical technologies for the communications port 90 include, but are not limited to, USB, IEEE 1934, Ethernet, Bluetooth or Wireless LAN. This would typically be in an inpatient situation.

An integral power source including an electrical cell in the form of rechargeable or single use or non-chargeable replaceable batteries are included (not shown) in the mask 10 to provide electrical power. In an alternative embodiment, the electrical power could be supplied by a suitable external power source. In another embodiment, solar power could be used, for example, through the use of photovoltaic cells.

The images can be transmitted to the medical professional in various forms and in a variety of ways. Thus, the images transmitted may be unprocessed, in which case, processing may occur remote from the subject. Alternatively, the images may be processed by the mask 10 prior to being sent to the medical professional. The level of processing may be varied and will be dictated by the features on the mask 10.

The manner in which the images are transmitted to the medical professional will vary depending on the functionality of the mask 10. When the mask 10 includes a removable storage means—such as the CF card 60—containing the data, the storage means could be sent to the health-care professional. When the mask 10 includes a data transmission means such as the communications port 90 described above, the images, processed or unprocessed, can be transmitted directly to the health care professional. This can be achieved via the Internet or any other suitable mode of data transmission.

When the mask 10 is adapted to produce a report, rather than transmit the images, the mask 10 may transmit the report to the medical professional.

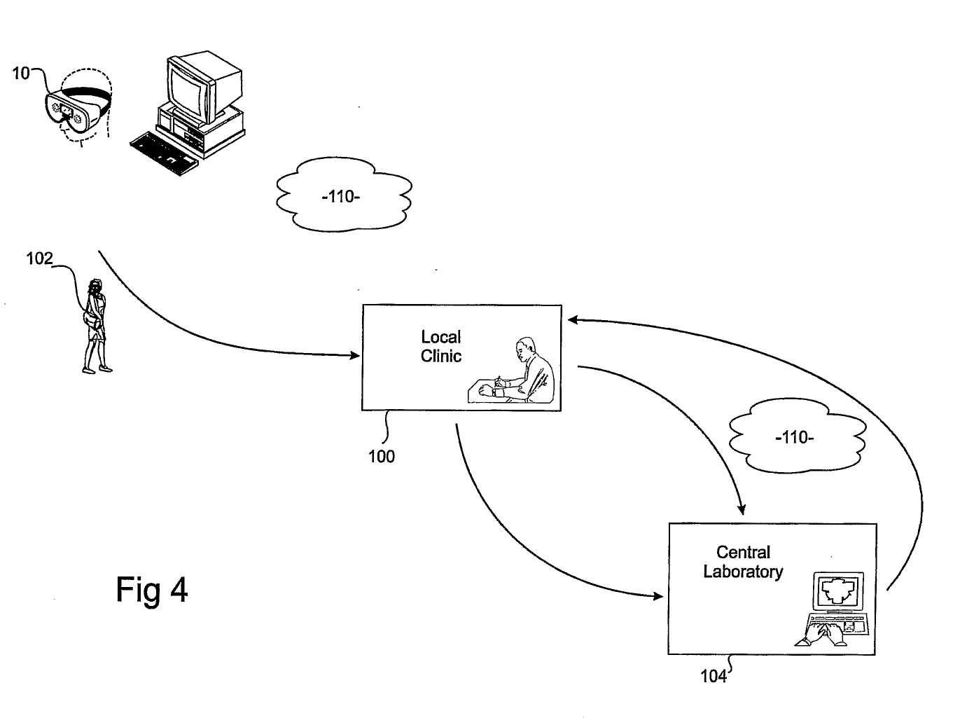

Preferred methods of investigating various neurological and other disorders using the mask 10 will now be described with reference to FIG. 4. In one preferred method of the invention, a clinic 100 will provide the mask 10 to a patient 102 under investigation for them to take home or with them where ever they go, that is in an outpatient scenario. The clinic 100 will also be responsible for training the patient or their carer to make themselves safe and to deploy the mask when they experience an attack of dizziness commencing. Deploying the mask involves placing the mask over the head in front of the eyes. The mask 10 is then held in place by the user, or using the strap 14. Activating the mask involves pressing a button for example on the user control interface 70, or applying a command via the communications port 90, that both activates the light source 20 to illuminate the patient's eyes 22 and the image sensors 40. The image sensors 40 will capture images of the eyes 22 for a predetermined period of time and will send these images to the IPU 50 for processing and storage in the storage media 60.

The mask 10 may subsequently be returned to the clinic 100 by the patient 102 for analysis of the recorded images. The clinic 100 can download the images using the communications port 90 or remove the CF card 60 and download the images from that. Alternatively, if the patient lives in a remote location, they may be able to download the images onto their home or remote clinic's personal computer 106 and transmit the compressed images to the clinic 100 via the Internet 110, or other suitable data communications network such as a wide area or local area network could be used.

The image analysis may be performed in the clinic 100 if it has the necessary specialised personnel. Alternatively, the images may be transmitted via the Internet to a central laboratory 104 for analysis by specialists. In the case of nystagmography the analysis concentrates on recorded eye movements during the dizzy attack. The analysis may make use of already existing eye tracking processing software which is able to analyse the eye images for vertical, horizontal or tortional eye movements, pupil diameter and other eye attribute parameters.

In this way, images can be taken at one location and transmitted to another location where they can be analysed and processed accordingly.

Another mode of use for the device is the real-time viewing and recording of eye images. This would be likely in an inpatient situation or in a situation where the data is transmitted wirelessly to a clinician or other health professional. This would involve a clinician or patient holding or attaching the device 10 to the patient's head in a local clinic 100. After processing the images the device 10 will send the images via the communications port 90 to a personal computer (PC) in the local clinic 100. The clinician can then choose to view, save, analyse and/or transmit these images to the central laboratory 104. The local clinic 100 may be a GP's clinic, a hospital emergency room, 24 hour medical clinic, remote clinic or specialist clinic, which has one or more of the devices 10 available in the clinic to perform a quick test.

In a more advanced version of the device, additional processing power is provided onboard the IPU 50 to perform programmed analysis of the stored images and to automatically generate a preliminary report that can be subsequently verified by a specialist if necessary. This is achieved by means of software that is operable to interrogate the stored data and issue the prescribed report. The report can then be transmitted using the communications port 90, instead of or in addition to the images.

Alternatively, suitable analysis software may be supplied to the clinic or user for use in a stand alone processing unit (such as a docking station for the device), or in a desktop PC. The stored images can then be downloaded from the mask 10 to the stand alone unit or PC via any suitable wireless or wired data communication path for automatic analysis.

Although the preferred embodiment of the device and method of use have focussed on the investigation of eye movement for vestibular and neurological disorders during the time of an episode, there are many other potential uses for the mask 10 including, but not limited to, the following:

(a) bedside monitoring of hospital inpatients;

(b) telemedicine applications for remote areas;

(c) monitoring the effect of drugs on the reduction of symptoms;

(d) wireless/hands free viewing of eye movements during postulography and manoeuvres such as Hallpike's manoeuvres;

(e) teaching tool;

(f) record keeping tool; and

(g) research tool.

A range of disorders can be monitored, diagnosed or excluded as a possible diagnosis using the method of the present invention including a range of dizziness and balance related disorders. The disorder may be one selected from the group consisting of: a vestibular system associated disorder or disease, Benign Paroxysmal Positional Vertigo (BPPV), Meniere's Disease, Secondary Endolymphatic Hydrops, Labyrinthitis, Vestibular Neuritis, Perilymph Fistula, Acoustic Neuroma, Ototoxicity, Vestibular Migraine, Mal de Debarquement, Pediatric Vestibular Disorders, Aging, Dizziness, and Balance Cervicogenic Dizziness, Otosclerosis, Cholesteatoma, Enlarged Vestibular Aqueduct, Vestibular Hyperacusis, Autoimmune Inner Ear Disease and Superior Canal Dehiscence.

The mask 10 may also be used to monitor the effectiveness of a treatment for a dizziness or balance related disorder. In this instance, monitoring the effectiveness of a treatment for a dizziness or balance related disorder comprises the steps of:
(i) applying the mask 10 to a subject undergoing treatment for the disorder when changes in eye movement are present;
(ii) capturing images of the subject's eyes 22 in digital form; and
(iii) analysing the images to monitor the effectiveness of the treatment.

The analysis step may include comparing the images to images of the same subjects eyes taken prior to treatment or earlier in the treatment regime when changes in the eye movement were present.

The treatments may be varied and include drug therapies, physical therapies and psychological therapies. Betahisitine dihydrochloride is one particular drug that is used to treat Meniere's Disease.

The mask 10 may also be used to monitor the effects of treatments that are capable of causing a dizziness or balance related disorder such as vestibular degeneration. In this instance, the method includes the steps of:
(i) applying the mask 10 to a subject undergoing treatment when changes in eye movement are present;
(ii) capturing images of the subject's eyes in digital form; and
(iii) analysing the images to monitor the effectiveness of the treatment.

Again the analysis step can comprise comparing the images to images of the same subjects eyes taken prior to treatment or earlier in the treatment regime when changes in the eye movement were present.

The treatments may be varied and include drug therapies, physical therapies and psychological therapies. Gentamcyin is one particular drug that is capable of causing vestibular degeneration and thus needs to be closely monitored when administered to subjects.

The analysis may also be aimed at diagnosing vestibular or neurological disorders. Typically the analysis will concentrate on recorded eye movements during an attack of dizziness or some other underlying cause of movement or changes to the physical characteristics of the eye(s). In particular the analysis may involve the identification of nystagmus during the time of a vestibular attack, as well as the intensity and direction of the nystagmus. Analysing eye movements during an attack of dizziness can be a key diagnostic tool in vestibular disorders. It may help determine whether the cause of a patient's imbalance problems is vestibular (inner ear) or otherwise, and if vestibular they may point towards a specific diagnosis.

The analysis of the captured images of changes in the eye may also help to diagnose other neurological disorders. Changes in other attributes of the eye such as discolouration, pupil size and iris size may also lead to diagnosis of numerous other medical conditions.

The properties of the mask 10 mean it can be applied in a range of situations to improve patient outcomes, particularly for subjects outside of the clinic or medical establishment. For example, the device can be taken home by a subject and used during a dizziness attack in their own home. The device is also particularly useful for patients with poor mobility and thus can also be used to carry out bedside assessments of patients in or out of hospital. Thus, the present invention also provides a system for the remote monitoring of patients, the system involving the use of the portable device of the present invention.

Another advantage of the portability of the mask 10 is that the subject has relatively free movement when wearing the device. Often it is useful to apply physical stimuli to the subject to create a dizziness attack. Some of these stimuli involve substantial movement of the subject that are hampered by wires or cords commonly associated with prior art apparatus. The portable device of the present invention can be conveniently worn when these stimuli are applied to ensure eye movement data is efficiently captured.

The mask 10 may also be used in for emergency room analysis. Studies on patients that present to an emergency rooms complaining of dizziness show that the most common cause (43%) of the symptom was peripheral vestibular disease. Thus, the present invention also provides a system for the monitoring of patients in the emergency room, the system involving the use of the portable device of the present invention.

As described above, the mask 10 records valuable data associated with a range of medical conditions. Once correlations between eye movement data recorded and actual medical disorders or conditions have been established, this can be stored and used as a valuable medical resource for future use. More particularly, when subjects use the device of the present invention the eye movement data obtained can be screened against a database containing a range of eye movement data that have been correlated with particular disorders or conditions. In so doing, indicative diagnoses can be made without any direct involvement of a medical practitioner. Thus, the present invention also provides a database containing an eye movement data correlated with a range of disorders and/or conditions.

A further use of the mask 10 is in a method of diagnosing a disease or disorder correlated with eye movement in a subject, where the method comprises the steps of:
(i) capturing images of the subject's eyes in digital form using the device described herein; and
(ii) comparing said images with a database of eye movement data correlated with a range of disorders and/or conditions to diagnose the disease or disorder.

The analysis step may include comparing images to images of the same subjects taken prior to treatment or earlier in a treatment regime when changes in the eye movement were present.

Variations are possible within the scope of the present invention and have been referred to above. The mask 10 may include some or all of the features described above. For example, the mask 10 may or may not include the communications port 90, the user control interface 70, and a removable storage means such as the CF card 60 and may include combinations of these features.

Those skilled in the art will appreciate that the invention described herein is susceptible to variations and modifications other than those specifically described. It is to be understood that the invention includes all such variations and modifications. The invention also includes all of the steps, features, compositions and compounds referred to and indicated in the specification, individually or collectively and any and all combinations or any two or more of the steps or features.

Now that preferred embodiments of the mask and method of use have been described in detail, it will be apparent that embodiments of the invention provide a number of advantages over the prior art, including the following:
(i) The mask is relatively inexpensive to manufacture and therefore can be made available at an affordable price to a broader clientele;
(ii) The mask is highly portable and therefore can be supplied to users to take home for self-administration;
(iii) It can be used in conjunction with a conventional desktop or laptop personal computer with no need for additional hardware and therefore reduces additional hardware costs;
(iv) It is self-contained, with the video images being able to be stored on board so that no external storage media are required;
(v) The mask is lightweight, durable and easy to hold and use.

1. A portable device adapted to cover the eyes of a subject in a manner that omits visible light, the device comprising:
light omission means arranged to substantially reduce visible light stimulation of the subject's eyes;
a non-visible light generating means to illuminate the subject's eyes;
a lens member for creating images of the subject's eye; and
an image capturing means for capturing the images in digital form.

2. A portable device according to claim 1, wherein the non-visible light is infrared light.

3. A portable device according to claim 1, wherein the light omission means comprises a light proof seal.

4. A portable device according to claim 1, wherein the lens member has a depth of field sufficient to accommodate different distances between various subject's eyes and the lens member.

5. A portable device according to claim 4, wherein the lens member has a fixed depth of field.

6. A portable device according to claim 4, wherein the lens member is adjustable.

7. A portable device according to claim 1, comprising a plurality of lens members.

8. A portable device according to claim 7, comprising two lens members, one for each eye.

9. A portable device according to claim 1, wherein the image capturing means comprises a digital image sensor.

10. A portable device according to claim 1, wherein the image capturing means comprises two digital image sensors, one for each eye.

11. A portable device according to claim 9, wherein the digital image sensor is capable of capturing at least 50-60 images per second.

12. A portable device according to claim 11, wherein the digital image sensor is capable of capturing at least 100-500 images per second.

13. A portable device according to claim 9, wherein the digital image sensor is a charge coupled device.

14. A portable device according to claim 9, wherein the digital image sensor is a complementary metal oxide semiconductor device.

15. A portable device according to claim 1, wherein the lens member and the image capturing means are provided integrally.

16. A portable device according to claim 15, wherein the integral lens member and image capturing means comprise a camera.

17. A portable device according to claim 15, comprising two integral lens members and image capturing means, one for each eye, and adapted to be synchronized to record images of both eyes at the same time.

18. A portable device according to claim 1, comprising an integral power source.

19. A portable device according to claim 18, wherein the integral power source includes an electrical cell.

20. A portable device according to claim 1, wherein the portable device is arranged to be coupled to a separate power source.

21. A portable device according to claim 1, wherein the portable device is of a size that enables it to be conveniently handheld.

22. A portable device according to claim 1, further comprising a data processing means for processing the captured images.

23. A portable device according to claim 22, wherein the data processing means includes a processing unit.

24. A portable device according to claim 23, wherein the data processing means includes two processing units, one for each eye.

25. A portable device according to claim 24, wherein the two processing units are operable in a Master/Slave configuration for processing the images from the left and right eye respectively.

26. A portable device according to claim 22, wherein the data processing means includes a tagging function to allocate a marker to the captured images.

27. A portable device according to claim 22, wherein the data processing means further comprises a communication means to communicate the status of at least one of the data processing means and the images contained therein.

28. A portable device according to claim 27, wherein the communication means includes an audio means for providing an audible indication to a user.

29. A portable device according to claim 27, wherein the communication means includes a visual display.

30. A portable device according to claim 27, wherein the communication means includes a user control interface.

31. A portable device according to claim 30, wherein the user control interface and the display means are provided integrally.

32. A portable device according to claim 22, wherein the data processing means includes a reporting function operable to provide reports based on interrogation of data processed by the data processing means.

33. A portable device according to claim 1, further including a data transmitting means for transmitting the captured images to a separate device.

34. A portable device according to claim 33, wherein the data transmitting means comprises a wireless data transmitter.

35. A portable device according to claim 33, wherein the data transmitting means comprises a wired data transmitter.

36. A portable device according to claim 33, wherein the data transmitting means is operable to transmit via the Internet.

37. A portable device according to claim 33, wherein the data transmitting means is operable to transmit in real-time.

38. A portable device according to claim 1, wherein the portable device further includes a data processing means for processing and storing the captured images, and data transmitting means operable to transmit the captured and stored images some time after they are captured.

39. A portable device according to claim 1, further including a data storage means for storing captured images thereon.

40. A portable device according to claim 39, wherein the data storage means comprises a removable storage device.

41. A portable device according to claim 39, wherein the data storage means is fixed within the portable device.

42. A method of assessing eye movement using the portable device of claim 1, the method comprising the steps of:
applying the portable device to a subject when changes in eye movement are present; and
capturing images of the subject's eyes in digital form.

43. A method according to claim 42, further including the steps of:
applying the device to the subject, at a first location, when changes in the eye may be present; and transmitting the images to a medical professional, at a second location, to assess the presence of a disorder.

44. A method according to claim 43, wherein the subject applies the portable device himself/herself during an episode in which changes in the eye may be present.

45. A method according to claim 43, wherein the transmitted images are unprocessed and the method includes the step of processing the transmitted images remotely from the subject.

46. A method according to claim 43, wherein the method includes the step of processing the images by the portable device prior to being sent to the medical professional.

47. A method according to claim 42, including the step of producing a report and transmitting the report to the medical professional.

48. A method of monitoring the effectiveness of a treatment for a dizziness or balance related disorder using the portable device of claim 1, the method comprising the steps of:
applying the portable device to a subject undergoing treatment for the disorder when changes in eye movement are present;
capturing images of the subject's eyes in digital foiiii; and
analyzing the images to monitor the effectiveness of the treatment.

49. A method according to claim 48, wherein the step of analyzing the images includes comparing the images to images of the same subject's eyes taken prior to treatment or earlier in the treatment regime when changes in the eye movement were present,

50. A method of monitoring the effects of a treatment capable of causing a dizziness or balance related disorder using the portable device of claim 1, the method comprising the steps of:
applying the portable device to a subject undergoing treatment when changes in eye movement are present;
capturing images of the subject's eyes in digital form; and
analyzing the images to monitor the effectiveness of the treatment.

51. A method according to claim 50, wherein the step of analyzing the images comprises comparing the images to images of the same subject's eyes taken prior to treatment or earlier in the treatment regime when changes in the eye movement were present.

52. A system for the bedside monitoring of patients, the system involving the use of the portable device of claim 1.

53. A system for the monitoring of patients in an emergency room, the system involving the use of the portable device of claim 1.

54. The portable device of claim 1, wherein the portable device transmits eye movement data to be screened against a database containing an eye movement data correlated with a range of at least one of disorders and conditions.

55. A method of diagnosing a disease or disorder correlated with eye movement in a subject using the portable device of claim 1, the method comprising the steps of:
capturing images of the subject's eyes in digital form using the portable device of claim 1; and
comparing said images with a database of eye movement data correlated with a range of at least one of disorders and conditions to diagnose the disease or disorder.

56. A portable device adapted to cover the eyes of a subject in a manner that omits visible light, the device comprising:
a non-visible light generating means to illuminate the subject's eyes;
a lens member for creating clear images of the subject's eye;
an image capturing means for capturing the images in digital form; and
a data processing means for processing the captured images.

57. A portable device adapted to cover the eyes of a subject in a manner that omits visible light, the device comprising:
a non-visible light generating means to illuminate the subject's eyes;
a lens member for creating clear images of the subject's eye;
an image capturing means for capturing the images in digital form; and
a data transmitting means for transmitting the captured images to a separate device.

58. A portable device adapted to cover the eyes of a subject in a manner that omits visible light, the device comprising:
a non-visible light generating means to illuminate the subject's eyes;
a lens member for creating clear images of the subject's eye;
an image capturing means for capturing the images in digital form;
a data processing means for processing the captured images; and
a data transmitting means for transmitting the captured images to a separate device.

59. A method of assessing eye movement using the portable device of claim 1, the method comprising the steps of:
applying the portable device of claim 1 to a subject, at a first location, when changes in the eye may be present;
capturing images of the subject's eyes in digital form using the portable device; and
transmitting said images to a medical professional, at a second location, to assess the presence of a disorder.

60. A portable device adapted to cover the eyes of a subject in a manner that omits visible light, the device consisting essentially of:
light omission means arranged to substantially reduce visible light stimulation of the subject's eyes;
a non-visible light generating means to illuminate the subject's eyes;
a lens member for creating images of the subject's eye; and
an image capturing means for capturing the images in digital form.

61. A method of assessing eye movement using the portable device of claim 60, the method consisting essentially of:
applying the device to a subject when changes in eye movement are present; and
capturing images of the subject's eyes in digital form.

62. A method of monitoring the effectiveness of a treatment for a dizziness or balance related disorder using the portable device of claim 60, the method consisting essentially of:
applying the portable device to a subject undergoing treatment for the disorder when changes in eye movement are present;
capturing images of the subject's eyes in digital form; and
analyzing the images to monitor the effectiveness of the treatment.

63. A method of monitoring the effects of a treatment capable of causing a dizziness or balance related disorder using a portable device of claim 60, the method consisting essentially of:
applying the portable device to a subject undergoing treatment when changes in eye movement are present;
capturing images of the subject's eyes in digital form; and
analyzing the images to monitor the effectiveness of the treatment.

64. A system for the bedside monitoring of patients, the system consisting essentially of the use of the portable device of claim 60.

65. A system for the monitoring of patients in an emergency room, the system consisting essentially of the use of the portable device of claim 60.

66. The portable device of claim 60, wherein the portable device transmits eye movement data to be screened against a database consisting essentially of an eye movement data correlated with a range of at least one of disorders and conditions.

67. A method of diagnosing a disease or disorder correlated with eye movement in a subject using the portable device of claim 60, the method consisting essentially of:
capturing images of the subject's eyes in digital form using the portable device; and
comparing said images with a database of eye movement data correlated with a range of at least one of disorders and conditions to diagnose the disease or disorder.

68. A portable device adapted to cover the eyes of a subject in a manner that omits visible light, the device consisting essentially of:
a non-visible light generating means to illuminate the subject's eyes;
a lens member for creating clear images of the subject's eye;
an image capturing means for capturing the images in digital form; and
a data processing means for processing the captured images.

69. A portable device adapted to cover the eyes of a subject in a manner that omits visible light, the device consisting essentially of:
a non-visible light generating means to illuminate the subject's eyes;
a lens member for creating clear images of the subject's eye;
an image capturing means for capturing the images in digital form; and
a data transmitting means for transmitting the captured images to a separate device.

70. A portable device adapted to cover the eyes of a subject in a manner that omits visible light, the device consisting essentially of:
a non-visible light generating means to illuminate the subject's eyes;
a lens member for creating clear images of the subject's eye;
an image capturing means for capturing the images in digital form;
a data processing means for processing the captured images; and
a data transmitting means for transmitting the captured images to a separate device.

71. A method of assessing eye movement using the portable device of claim 60, the method consisting essentially of:
applying the portable device to a subject when changes in eye movement are present; and
capturing images of the subject's eyes in digital form.

72. (canceled)

73. (canceled)

74. (canceled)

75. (canceled)

76. (canceled)
